(19) United States
(12) Patent Application Publication
(10) Pub. No.: US 2018/0360025 A1
(43) Pub. Date: Dec. 20, 2018

(54) ARTICLE OF MANUFACTURE AND METHODS FOR INCREASING SURVIVAL OF RED BLOOD CELLS

(71) Applicants: Ramot at Tel-Aviv University Ltd., Tel-Aviv (IL); The Medical Research, Infrastructure and Health Services Fund of the Tel Aviv Medical Center, Tel-Aviv (IL)

(72) Inventors: Rina ROSIN-ARBESFELD, Herzlia (IL); Ronen SIMAN-TOV, Tel-Aviv (IL)

(21) Appl. No.: 15/739,857

(22) PCT Filed: Jul. 21, 2016
(86) PCT No.: PCT/IL2016/050804

§ 371 Date: Dec. 26, 2017

Related U.S. Application Data
(60) Provisional application No. 62/195,347, filed on Jul. 22, 2015.

(51) Int. Cl. A01N 1/02; A61K 31/522

(52) U.S. Cl. CPC A01N 1/0263; A01N 1/0242; A01N 1/0226; A61K 31/522

(57) ABSTRACT

An ex-vivo method of increasing survival of red blood cells (RBCs) is provided. The method comprising contacting the RBCs with an activator of the non-canonical Wnt pathway, which results in actin polymerization, thereby increasing survival of red blood cells (RBCs). Also provided is a method of storing RBCs and treating a medical condition associated with RBC cytoskeleton/membrane disease.

### FIELD AND BACKGROUND OF THE INVENTION

The present invention, in some embodiments thereof, relates to an article of manufacture and methods for increasing survival of red blood cells.

The Wnt glycoproteins are involved in multiple aspects of embryonic development and adult homeostasis. Depending on the type of ligand-receptor interaction, the availability of intracellular signaling components and the specific target cell, the Wnt cascades can be divided into different pathways. In the non-canonical Wnt pathway, specific Wnt-Frizzled receptor interactions induce actin cytoskeleton alterations through the activity of the Rac GTPases. These Rac proteins are important for stabilization of the anucleated red blood cell (RBC) cytoskeleton.

RBCs are anucleated cells that function as oxygen carriers and can survive up to 120 days in the bloodstream. Massive loss of these cells, through trauma or disease, is a life-threatening condition mainly treated by blood transfusions of stored blood. Thus, RBC storage is of great importance. Currently, RBCs can be stored ex-vivo up to 42 days, during these storage period, they undergo senescence and gradually lose their ability to carry oxygen.

Previous studies have shown that some of the non-canonical Wnt signaling components, such as Rac1 and protein kinase C (PKC) are present in RBC's, and are involved in stabilization of the cells cytoskeleton complex (Goodman S R. Exp Biol Med. 2007 232(11):1391-408).

Interestingly, though, Wnt polypeptides are not expressed in RBCs.

Several studies demonstrate that Rac1 and Rac2 deficiency causes anemia with reticulocytosis, indicating decreased RBC deformability and survival. Erythrocytes deficient of Rac1 and Rac2 GTPases exhibit altered actin assembly in membrane skeleton. In these erythrocytes, adducin is hyper-phosphorylated by PKC, leading to decreased F-actin capping at the barbed ends, dissociation of spectrin from actin, and increased fragility of the RBC cytoskeleton (Kalfa T A. Blood. 2006 108(12):3637-45). As a result F-actin aggregates and actin-to-spectrin ratio in the membrane is increased, indicating weaker association to the membrane skeleton (Kalfa , supra).

Additional background art includes:

U.S. Patent Application Publication Number 20080226707 teaches Wnt proteins, where the Wnt protein is inserted in the non-aqueous phase of a lipid structure. The Wnt protein may be used for accelerating bone marrow repair and treatment of bone disease and injuries via an effect on bone stromal cells.

U.S. Patent Application Publication Number 20060147435 discloses methods for increasing stem cells, hematopoietic progenitor/stem cells, mesenchymal progenitor/stem cells, mesodermal progenitor/stem cells, muscle progenitor/stem cells, or neural progenitor/stem cells in vivo in a mammalian subject. Methods are also provided for treating an immune related disease, a mesenchymal/mesoderm degenerative disease, or a neurodegenerative disease in a mammalian subject in need thereof. Also taught are methods of treating immunodeficiency disease, for example, primary immunodeficiency, such as ADA-deficient SCID, X-linked SCID, common variable immunodeficiency, chronic granulomatous disease (CGD), X-linked agammaglobulinemia, Wiskott-Aldrich syndrome; hemoglobinopathy, such as sickle cell anemia, .beta-thalassemia; other single-gene disorders, such as Hurler's disease, Gaucher's disease, hemophilia A, hemophilia B, .alpha.-1 antitrypsin deficiency; or stem cell defects, such as Fanconi anemia.

U.S. Pat. No. 5,851,984 discloses Wnt polypeptides in hematopoiesis. In particular, in vitro and in vivo methods for enhancing proliferation or differentiation of a hematopoietic stem/progenitor cell using a Wnt polypeptide, and optionally another cytokine. Specifically disclosed are methods of increasing erythropoiesis using Wnt polypeptides

U.S. Pat. No. 6,159,462 discloses uses for Wnt polypeptides in hematopoiesis. In particular taught are in vitro and in vivo methods for enhancing proliferation, differentiation or maintenance of a hematopoietic stem/progenitor cell using a Wnt polypeptide, and optionally another cytokine.

### SUMMARY OF THE INVENTION

According to an aspect of some embodiments of the present invention there is provided an ex-vivo method of increasing survival of red blood cells (RBCs), the method comprising contacting the RBCs with an activator of the non-canonical Wnt pathway, which results in actin polymerization, thereby increasing survival of red blood cells (RBCs).

According to an aspect of some embodiments of the present invention there is provided a method of storing RBCs, the method comprising contacting the RBCs with an activator of the non-canonical Wnt pathway, which results in actin polymerization, thereby storing red blood cells (RBCs).

According to an aspect of some embodiments of the present invention there is provided a population of cells obtainable according to the method described herein.

According to an aspect of some embodiments of the present invention there is provided a method of treating a medical condition associated with RBC cytoskeleton/membrane disease, the method comprising administering to a subject in need thereof a therapeutic effective amount of an activator of the non-canonical Wnt pathway, which results in actin polymerization, thereby treating the medical condition associated with RBC cytoskeleton/membrane disease.

According to an aspect of some embodiments of the present invention there is provided an article of manufacture comprising a blood container (e.g., blood bag) comprising an activator of the non-canonical Wnt pathway, which results in actin polymerization.

According to an aspect of some embodiments of the present invention there is provided A population of cells obtainable according to the methods as described herein.

According to an aspect of some embodiments of the present invention there is provided use of the population of cells for RBC transfusion in a subject in need thereof.

According to some embodiments of the invention, the article of manufacture further comprises a preservative and/or an anticoagulant.

According to some embodiments of the invention, said RBCs are comprised in whole blood.

According to some embodiments of the invention, said RBCs are comprised in a population of cells comprising platelets.

According to some embodiments of the invention, said RBCs are comprised in a population of cells devoid of platelets.

According to some embodiments of the invention, said RBCs are comprised in a population of cells devoid of nucleated blood cells.

According to some embodiments of the invention, said RBCs are comprised in a population of cells devoid of bone marrow cells.

According to some embodiments of the invention, said bone marrow cells comprise mesenchymal stem cells. According to some embodiments of the invention, said RBCs are a pure population of cells (i.e., substantially 100% of the contacted cells are RBCs).

According to some embodiments of the invention, said RBCs comprise irradiated RBCs.

According to some embodiments of the invention, said RBC cytoskeleton/membrane disease is selected from the group consisting of spherocytosis, elliptocytosis, pyropoikilocytosis and stomatocytosis.

According to some embodiments of the invention, said component of the non-canonical Wnt pathway comprises Wnt.

According to some embodiments of the invention, said Wnt is selected from the group consisting of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11 and Wnt16.

According to some embodiments of the invention, said Wnt is Wnt5A.

According to some embodiments of the invention, said Wnt is Wnt3A.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C show increased survival and basic parameters of RBCs treated with Wnt3a and Wnt5a CM: Fresh RBCs were incubated with control (C), Wnt3a (3) or Wnt5a CM for the indicated time periods at 37° C. (A) The RBCs were viewed and photographed (X20) (B) The number of the RBCs was counted using a hemocitometer. (C) RBCs from B were analyzed for: mean cellular volume (MCV), hemoglobin (HGB) and hematocrit levels.

FIG. 2 shows that Wnt ligands protect the RBCs membrane: treating RBCs with Wnt CM leads to maintained morphology of the cells membrane. Note the spiky appearance of many cells in the control medium (indicated by arrows). Cells were sedimented using a shandon cytospin before fixation and staining.

FIGS. 3A-B show hemoglobin levels and RBCs morphology are affected by Wnt ligands. RBCs treated with Wnt-3a or Wnt-5a CM at 37° C. for 24 h were stained for hemoglobin and actin. The figures show shrunken, irregular and “spiky” shaped RBCs (arrow) with lower hemoglobin levels in the control cells. Similar results were obtained with Wnt-5a. Actin also seems to aggregate in around 50% of the control cells.

FIG. 4 shows the effects of Wnt ligands on RBC proteins. Fresh RBCs were treated with the Wnt ligands as indicated. Cells were separated to cytoplasmic and membrane fractions 24 h later. Western blot analysis was performed using specific antibodies as indicated. Shown is the membrane fraction unless specifically indicated. Please note that Wnt treatment leads to shift of actin from the cytoplasm to the membrane, and increased levels of PKC, Rac1 and p-JNK.

FIG. 5 is an image of the canonical and non-canonical Wnt pathways (adopted from Hitt, E. (2013) Wnt signaling inhibition: Will decades of effort be fruitful at last? global.onclive.com/publications/oncology-live/2012/december-2012/wnt-signaling-inhibition-will-decades-of-effort-be-fruitful-at-last. The Wnt/Ca²⁺ pathway increase intracellular calcium levels and mediate other signaling pathways. The Wnt PCP pathway initiates a cascade that activates the Rac and Rho GTPases to mediate cell polarity, cell survival and cytoskeleton rearrangement by actin modification.

FIG. 6 is a graph showing mRNA expression of Wnt signaling components in Reticulocytes. Red bars: Wnt/canonical pathway; Wnt-3A, β catenin. Green bars: Wnt/PCP pathway; disheveled associated activator of morphogenesis DAAM1, JNK1, RHOA. Blue bars: Wnt/calcium pathway; NFATC1 (calcineurin-dependent 1).

FIG. 7 showing expression of the membrane proteins of the Wnt signaling pathway in treated RBCs. Fresh RBCs were treated with the Wnt ligands as indicated. Cells membrane fraction was separated 16 h later. Western blot analysis was performed using specific antibodies as indicated. All RBC shown Fzd and DVL protein expression, specifically Fzd1 and Dvl-2 (also phosphorylated Dvl-2). Treated RBC with Wnt-3a and Wnt-5a shown Wnt-3a and Wnt-5a expression, respectively.

FIG. 8: mRNA expression of Wnt signaling components in Reticulocytes. Wnt signaling array was used to identify the mRNA of these proteins in pre mature erythrocyte; Black bars: Wnt-3a. Blue bars: Fzd (1-9). Red bars: LRP5, LRP6. Green bars: DVL1, DVL2.

FIG. 9 shows mRNA expression of Wnt signaling components in Reticulocytes. Wnt signaling array was used to identify the mRNA of these proteins in pre mature erythrocyte; Black bars: Wnt-3a. Blue bars: Fzd (1-9). Red bars: LRP5, LRP6. Green bars: DVL1, DVL2.

FIG. 10 shows protein expression of the non-canonical Wnt signaling in treated RBC. In order to examine the effects of Wnt ligands on RBC proteins, fresh RBCs were treated with the Wnt ligands as indicated. Cells were separated to cytoplasm and membrane fractions 16 h later. Western blot analysis was performed using specific antibodies as indicated. Shown is the membrane fraction. Wnt treatment leads to increase levels of PKC, active Rac1, p-JNK and RhoA.

FIG. 11 shows actin distribution in RBCs after treatment with Wnts CM. Wnt treatment leads to shift of actin from the cytoplasm to the membrane. RBCs treated with Wnt-3a or Wnt-5a CM at 37° C. for 24 h to visualize hemoglobin and actin distribution, cytoskeleton structure and RBC morphology. The cells were stained filamentous actin (actin F). The figure shows that the number of cells which stained for membrane actin was significantly higher after treating the cells with Wnts condition media. Images were obtained with a X 60 and X20 oil-immersed objective lens after 24 hours incubation. Scale bar represents 5 μm and 25 μm respectively.

FIGS. 12A-B shows that Wnt-3a and Wnt-5a CM rebuild the cytoskeleton membrane in CytoD-treated RBC as demonstrated by Wright Gimesa staining (FIG. 12A) or immunofluorescence with anti-phalloidin antibody for filamentous actin (Abcam 176753) and anti-Hemoglobin antibody (Santa Cruz 21005) (FIGS. 12B).

FIG. 13 is an image showing cytoskeletal protein expression in treated RBCs. Left panel: adducin, and phospho adducin were detected by immunoblotting of cells harvested after 16 hours. Cells treated with wnt3a and Wnt5a conditioned media showed no change in adducin levels, while the phosphorylated forms of adducin both S724 and T445 were increased significantly.

FIG. 14: Effect of Wnts on the osmotic fragility of human red cells. Blood samples were incubated with control medium, wnt3a and Wnt5a condition medium for 6 hours in hypotonic solution. The hemolysis percentage was calculated for each NACL concentration. The upper right figure shows the hemolysis after each treatment before “fragility curves” were drawn. The hemolysis in the Wnt-3a and Wnt-5a mediums are lower in comparison to the control samples.

### DESCRIPTION OF SPECIFIC EMBODIMENTS OF THE INVENTION

The present invention, in some embodiments thereof, relates to an article of manufacture and methods for increasing survival of red blood cells. Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have now uncovered that treating red blood cells (RBCs) with Wnt-3a or Wnt-5a increases the survival and efficacy of RBCs and alters their protein expression pattern. The changes include increase in Rac1 and phospho JNK protein levels and a reduction in cytoplasmic actin levels. It is suggested that Wnt-3a and Wnt-5a initiate the non-canonical Wnt pathway in RBC leading to the activation of Rac1. This activation can affect the cells cytoskeleton in two ways: the first by actin polymerization and the second by inhibiting the breaking down of the cytoskeleton complex (actin-spectrin-adducin). Eventually, these two lead to stabilization of the cytoskeleton complex by actin modifications which are essential for RBC survival and their function as oxygen carriers.

The present inventors also found that activators of the non-canonical Wnt pathway reduce hemolysis, prevent osmolarity mediated fragility of RBCs, alleviate cytoplasmic perforation following CytoD treatment and affect actin cellular distribution (from a cytoplasmic distribution to a membrane distribution).

The present inventors have thus uncovered a novel function of the Wnt ligands in RBCs.

Of note, although Wnt3a is a canonical Wnt, in RBCs it seems to bind and activate the non-canonical Wnt pathway.

These findings may lead to increasing the survival and efficiency of RBCs, for prolonging storage condition or novel therapeutic strategies for people suffering from hemolytic disease such as anemia.

Thus, according to an aspect of the invention there is provided an ex-vivo method of increasing survival of red blood cells (RBCs), the method comprising contacting the RBCs with an activator of the non-canonical Wnt pathway, which results in actin polymerization, thereby increasing survival of red blood cells (RBCs).

As used herein “survival” refers to an increase in cell viability of an ex vivo RBC sample through a predetermined time. As used herein “increase” refers to at least 20%, increase, 30%, increase, 40%, increase, 50%, increase, 60%, increase, 70%, increase, 80%, increase, 90%, increase or even 100%, 200%, 300% increase in cell survival as compared to the same under identical conditions in the absence of the activator of the non-canonical Wnt pathway.

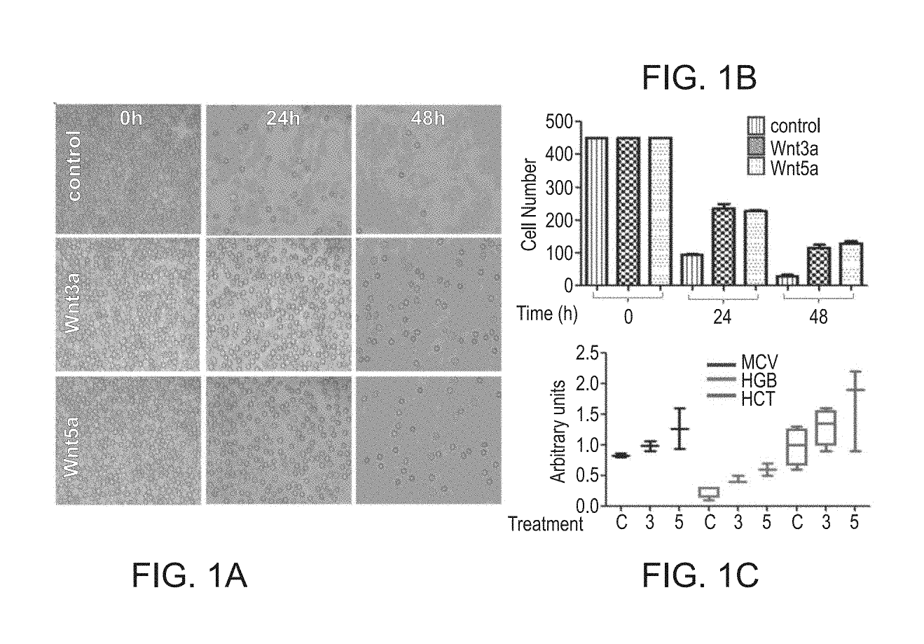

As shown in FIG. 1A and FIG. 1B cell survival can be monitored using methods which are well known in the art, including cell counting.

As used herein a “predetermined time” refers to at least minutes or hours e.g., 24-48 h. However, longer time periods also contemplated which are relevant for RBC storage are provided hereinbelow.

According to an alternative or an additional aspect there is provided a method of storing RBCs, the method comprising contacting the RBCs with an activator of the non-canonical Wnt pathway, which results in actin polymerization, thereby storing red blood cells (RBCs).

As used herein “contacting” refers to contacting with a soluble agent (e.g., in a solution) or an immobilized agent. For instance, the activator of the non-canonical Wnt pathway can be incorporated into the surface (coated) or into the base material of laboratory or hematology equipment such as blood collection or storage devices, a conduit, a flask, a bottle, a dish, a petri dish, a plate, a multi-well plate, a test tube, a blood transfusion bag, and other devices that are designed to contact blood or fluids comprising blood in-vitro.

According to an alternative or an additional aspect there is provided an article of manufacture comprising a blood container (e.g., bag) comprising an activator of the non-canonical Wnt pathway, which results in actin polymerization.

As used herein “red blood cells” also referred to as erythrocytes, reticulocytes or herein abbreviated as RBCs are the most common type of blood cell and the vertebrate organism's principal means of delivering oxygen (O₂) to the body tissues—via blood flow through the circulatory system. RBCs take up oxygen in the lungs or gills and release it into tissues while squeezing through the body's capillaries. The cytoplasm of RBCs is rich in hemoglobin. The cell membrane is composed of proteins and lipids, and this structure provides properties essential for physiological cell function such as deformability and stability while traversing the circulatory system and specifically the capillary network. The present inventors have now uncovered that RBCs express non-canonical Wnt pathway components to a higher level than the canonical Wnt pathway.

According to a specific embodiment, the blood cells are human red blood cells. However, veterinary applications are also contemplated.

For ex-vivo applications, red blood cells can be obtained from whole blood by centrifugation, which separates the cells from the blood plasma in a process known as blood fractionation. Packed red blood cells, which are made in this way from whole blood with the plasma removed, are used in transfusion medicine.

It will be appreciated that the red blood cells may comprise plasma or may be washed and used without the plasma component.

Alternatively or additionally, the RBCs are comprised in a whole blood (e.g., non-separated from other populations of cells that are present in the blood e.g., white blood cells, platelets).

Alternatively or additionally, the RBCs are comprised in a population of cells comprising platelets. Such a population is typically devoid of white blood cells.

Alternatively or additionally, the RBCs are comprised in a population of cells devoid of platelets. Such a population may be devoid of white blood cells. According to an alternative embodiment, such a population may comprise white blood cells.

According to a specific embodiment, the RBCs constitute about 100% of the population of cells.

According to a specific embodiment, the sample is which comprises the RBCs is essentially all non-nucleated cells (i.e., about 100%).

Accordingly, the RBCs are comprises in a population of cells devoid of nucleated blood cells (e.g., less than 10%, 5% or even 1%).

According to a specific embodiment, the RBCs are devoid of the buffy coat and plasma.

According to a specific embodiment, the RBCs comprise the buffy coat but is devoid of plasma.

According to a specific embodiment, the RBCs are comprised in a population of cells devoid of bone marrow cells.

As used herein “bone marrow cells” refers to the cells which reside in the bone marrow. Aside from differentiated cells (such as fibroblasts), these cells also comprise hemopoietic stem cells (which can produce blood cells) and mesenchymal stem (stromal cells, MSCs, which can produce fat, cartilage and bone).

Thus since bone marrow cells comprise mesenchymal stem cells, the present teachings also contemplate in a specific embodiment, RBCs that are comprised in a sample which is devoid of mesenchymal stem cells (other bone marrow components may be present in certain embodiments, however complete absence of bone marrow components is contemplated).

The International Society for Cellular Therapy is assuring MSC identity by using CD70, CD90, and CD105 as positive markers and CD34 as a negative marker.

Additional methods and markers for identification of MSCs are described in Lin et al. Histol Histopathol. 2013 Septmeber;28(9):1109-16, which is hereby incorporated by reference in its entirety.

As used herein “devoid” or “absent” refers to a level of below 10%, 5%, or even 1%, as determined by known molecular biology, FACs, or marker assays.

It will be appreciated that the RBCs can be purified from a subject of interest (autologous or from a donor subject i.e., non-autologous) provided measures are taken to avoid blood-group non-compliance.

Alternatively or additionally, the RBCs are ex-vivo differentiated from stem cells. Non-limiting examples of such protocols are known in the art. One such a protocol is provided in “First red blood cells grown in the lab,” New Scientist News, 19 August 2008, which is hereby incorporated by reference in its entirety.

According to a specific embodiment, the RBCs are a pure population of cells i.e., essentially or substantially all, 100%, of the contacted cells are RBCs.

According to a specific embodiment, the RBCs are selected from the group consisting of:
1. RBC concentrates.
2. RBC concentrates deprived of the buffy coat.
3. RBC concentrates with additive solutions (as further described hereinbelow).
4. RBC concentrates deprived of the buffy coat and resuspended in additive solutions.
5. Washed RBC.
6. Leucodepleted RBC.
7. Frozen RBC.
8. Apheretic RBC.
9. Irradiated RBC.

As used herein “an activator of the non-canonical Wnt pathway” refers to a molecule which upon contacting with RBCs results in actin polymerization via the activation of the non-canonical Wnt pathway.

The activator may act extracellularly by binding a receptor on the cell (Frizzeled), or intracellularly by acting downstream in the Wnt pathway. In the latter case the activator has a chemistry or is modified by a chemistry which renders it cell penetratable, while retaining its activity. In the first case, where the activator acts extracellularly it may be further modified to be protected from the hydrolytic functions of the serum in in-vivo applications. An activator may be modified by any one or more such modifications.

According to an alternative or an additional embodiment, the activator of the non-canonical Wnt pathway may mediate at least one of (two of, or all of) increased survival, reduction of hemolysis, morphology preservation, prevention of osmolarity mediated fragility of RBCs, alleviation of cytoplasmic perforation following CytoD treatment and shift of actin cellular distribution (from a cytoplasmic distribution to a membrane distribution).

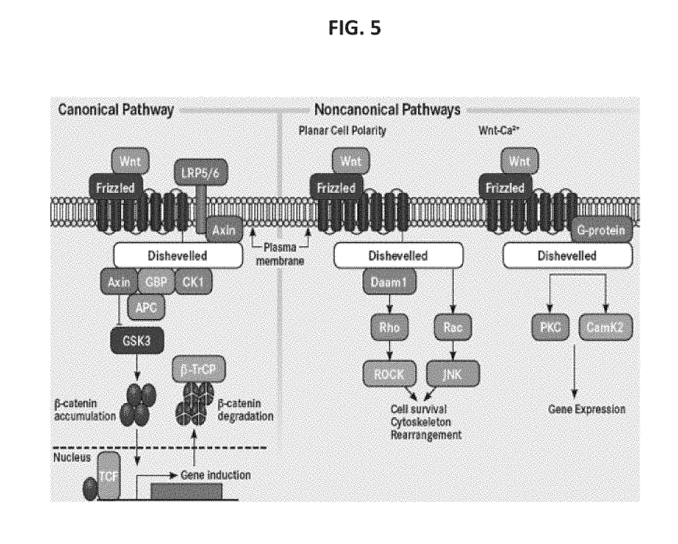

As used herein “the non-canonical Wnt pathway” refers to the signaling pathway, components of which are illustrated in FIG. 5. Examples include, but are not limited to Wnt, Frizzled, Dishevelled, Daam1, Rho, Rac, Rock, Jnk, PKC and CamK2.

The non-canonical pathway is often referred to as the β-catenin-independent pathway. This pathway can be further divided into at least two distinct branches, the Planar Cell Polarity pathway (or PCP pathway) and the Wnt/Ca2+ pathway, of which only the PCP is discussed in further detail herein, but both are contemplated according to the present teachings. The PCP pathway emerged from genetic studies in Drosophila in which mutations in Wnt signaling components including Frizzled and Dishevelled were found to randomize the orientation of epithelial structures including cuticle hairs and sensory bristles. Cells in the epithelia are known to possess a defined apical-basolateral polarity but, in addition, they are also polarized along the plane of the epithelial layer. This rigid organization governs the orientation of structures including orientation of hair follicles, sensory bristles and hexagonal array of the ommatidia in the eye. In vertebrates, this organization has been shown to underlie the organization and orientation of muscle cells, stereo-cilia in the sensory epithelium of the inner ear, the organization of hair follicles, and the morphology and migratory behavior of dorsal mesodermal cells undergoing gastrulation.

Wnt signaling is transduced through Fz independent of LRP5/6 leading to the activation of Dsh. Dsh through Daaml mediates activation of Rho which in turn activates Rho kinase (ROCK). Daaml also mediates actin polymerization through the actin binding protein Profilin. Dsh also mediates activation of Rac, which in turn activates INK. The signaling from Rock, JNK and Profilin are integrated for cytoskeletal changes for cell polarization and motility during gastrulation.

Wnts that can signal through the non-canonical Wnt signaling pathway include, but are not limited to Wnt1, Wnt2, Wnt2B, Wnt4, Wnt5A, Wnt5B, Wnt6, Wnt7A, Wnt7B, Wnt8A, Wnt8B, Wnt9A, Wnt9B, Wnt10A, Wnt10B, Wnt11 and Wnt16.

According to a specific embodiment, the component of the Wnt pathway refers to the planar cell polarity pathway e.g., Wnt, Frizzled, Dishevelled, Daam1, Rho, Rac, Rock, Jnk.

According to a specific embodiment, the non-canonical Wnt pathway leads to increased cell survival and cytoskeleton rearrangements as well as at least one of reduction of hemolysis, morphology preservation, prevention of osmolarity mediated fragility of RBCs, alleviation of cytoplasmic perforation following CytoD treatment and shift of actin cellular distribution (from a cytoplasmic distribution to a membrane distribution).

The activity of the molecule may be upstream or downstream in the pathway, e.g., Wnt binds Frizzled and therefore is an upstream component. Further downstream components are considered as activators of Rac, JNK phosphorylation, actin polymerization. Contemplated are proteinaceous products (e.g., Wnt polypeptides), as well as small molecules, e.g., activators of JNK phosphorylation. The specification (see Examples section below) provides numerous assays for qualifying molecules suitable for use according to the present teachings. These may comprise functional assays (e.g., effect on RBC survival, osmolarity mediated fragility, hemolysis etc.) and./or structural assays (e.g., signaling assays e.g., actin polymerization, phosphorylation e.g., Jnk, adducin, expression). According to a specific embodiment, a molecule suitable for use according to the present teachings is qualified by both activation of the non-canonical Wnt pathway and by a functional effect on RBCs such as provided hereinabove (increased survival, reduction of hemolysis, morphology preservation, prevention of osmolarity mediated fragility of RBCs, alleviation of cytoplasmic perforation following CytoD treatment and shift of actin cellular distribution (from a cytoplasmic distribution to a membrane distribution).

According to a specific embodiment, the activator of the non-canonical Wnt pathway comprises a small molecule.

According to a specific embodiment, the activator of the non-canonical Wnt pathway comprises a polypeptide agonist, also referred to herein as peptide.

According to a specific embodiment, the polypeptide agonist comprises Wnt.

According to a specific embodiment, the Wnt is selected from the group consisting of Wnt1, Wnt2, Wnt2B, Wnt4, Wnt5A, Wnt5B, Wnt6, Wnt7A, Wnt7B, Wnt8A, Wnt8B, Wnt9A, Wnt9B, Wnt10A, Wnt10B, Wnt11 and Wnt16.

According to a preferred embodiment, the Wnt is Wnt5A.

According to a specific embodiment, the Wnt is Wnt3A.

Wnt polypeptides are available commercially (e.g., R&D systems) and may be used as purified, non-purified (conditioned medium), or recombinant factors.

Also contemplated herein are Wnt polypeptides (or nucleic acid sequences encoding same for recombinant production) having modification(s) while retaining their effect on the RBCs, according to the present teachings.

Thus for example, EP 2663576, which is hereby incorporated by reference in its entirety teaches Wnt polypeptides having the amino acid sites of lipidation altered so that no post-translational lipidation occurs. The proteins retain Wnt biological activity, and the invention thus provides modified Wnt compositions having improved biologic drug-like properties such as enhanced solubility, production, and formulation, and therapeutic uses for such Wnt compositions. Also taught are Wnt polypeptides, including fusion polypeptides, that are suitable for clinical scale production and therapeutic use.

As used herein the terms “retaining” or “ aintaining,” or “retain” or “maintain”, generally refer to the ability of a Wnt composition of the invention to produce or cause a physiological response(i.e., measurable downstream effect) that is of a similar nature to the response caused by a Wnt composition of the naturally occurring Wnt amino acid or nucleic acid sequence. For example, the Wnt compositions of the invention exhibit Wnt biological activity of increasing survival of RBCs in vitro. Thus, a modified Wnt of molecule that retains the activity of the non-canonical Wnt pathway produce a. physiological response, such as survival of RBCs, that is of a similar nature to the response caused by a naturally occurring Win polypeptide. For example, a modified Wnt polypeptide elicits a similar physiological response that is at least 5%>, at least 10%>, at least 15%>, at least 20%>, at least 25%>, at least 30%, at least 40%, at least 50%, at least 60%, at least 70%, at east 75%, at least 80%, at least 85%, at least 90%, at least 95% or about 100% of the level of physiological response elicited by a composition comprising a naturally occurring Wnt amino acid or nucleic acid sequence.

According to a specific embodiment, the activator is a recombinant produced molecule.

As mentioned, and according to specific embodiments, a peptide activator (e.g., Wnt) may comprise modifications and/or additions which, for example, render the peptides even more stable while in a body or under ex-vivo conditions and/or more capable of penetrating into cells. According to specific embodiments the polypeptide comprises a modification and/or addition selected from the group consisting of N terminus modification, C terminus modification, peptide bond modification, modified amino acid, non-natural amino acid, a non-proteinaceous moiety and a penetrating agent.

As used herein, the term “modification” refers to the peptide wherein at least one of its amino acid residues is modified either by natural processes, such as processing or other post-translational modifications, or by chemical modification techniques which are well known in the art. Such modifications include, but are not limited to N terminus modification, C terminus modification, peptide bond modification, backbone modifications, and residue modification. Among the numerous known modifications typical, but not exclusive examples include: acetylation, acylation, amidation, ADP-ribosylation, glycosylation, glycosaminoglycanation, GPI anchor formation, covalent attachment of a lipid or lipid derivative, methylation, myristlyation, pegylation, prenylation, phosphorylation, ubiqutination, or any similar process. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Contacting may be performed on blood that has been stored or may be performed on “fresh” blood (or fractions thereof as described above in great detail) at a time proximate to the time it is withdrawn from the subject e.g., donor. Accordingly, a “non-stored” blood is subjected to the treatment at any time 24 hours or less after an initial event (e.g., donation of blood or self-donation), such as concurrent with the event, or 24 hours, 18 hours, 12 hours, 6 hours, 4 hours, 2 hours, 1 hour, 30 minutes, 15 minutes, 10 minutes, 2 minutes, 1 minute, or less, after the initial event.

The volume of blood can be concentrated, by removal of at least a portion of blood plasma, to produce red blood cell concentrate (RBCC). Storage may be, for example, from 1 to 50 days, or longer, as needed. Storage may be at any temperature and other conditions so as to maintain viability of the RBCs for clinically acceptable storage period. For example, storage may be at a temperature of from about 1° C. to about 6° C. In other embodiments, the RBCs may be frozen, at a temperature of about −65° C. or lower, with the addition of preservatives that preserve the viability of the RBCs at such low temperatures. Suitable preservatives include for instance cryopreservatives, including glycerol. It is understood that such preservative agents are to be removed from the RBCs prior to administration.

Other anticoagulant and preservatives (such as, for example Citrate Phosphate Dextrose Adenine (CPDA), Citrate Phosphate Dextrose (CPD), or heparin) can be included in the composition comprising the activator of the non-canonical Wnt pathway or as a separate formulation from the activator described herein (also referred to herein as an article of manufacture.

According to a specific embodiment, the contacting is effected for a time sufficient to allow RBCs in the blood to assimilate the activator of the non-canonical Wnt pathway and achieve a desired effect on a biochemical or biomechanical function of RBCs.

The skilled artisan would know how to modify various parameters during contacting including, time of contacting, the use of mechanical agitation and temperature of the blood during incubation. Mixing can be performed by swirling, shaking, rotating, or agitating.

In some embodiments, the blood is tested during incubation to determine whether one or more desired biochemical or biomechanical attributes of the RBCs have been attained (including functional assays for increased survival, reduction of hemolysis, morphology preservation, prevention of osmolarity mediated fragility of RBCs, alleviation of cytoplasmic perforation following CytoD treatment and shift of actin cellular distribution (from a cytoplasmic distribution to a membrane distribution).

It will be appreciated that such monitoring can also be effected when the non-canonical Wnt pathway activator is administered in vivo. Accordingly the subject may be tested for RBC function and or other biochemical properties (prior to and/or following treatment with the activator of the non-canonical Wnt pathway or RBCs treated with same).

The present teachings also contemplate a population of cells comprising RBCs having been treated (obtained or obtainable) using the present teachings.

The present invention also relates to methods for administering (e.g., transfusing) such a population of cells to a subject in need thereof. Such methods comprise any procedure suitable for administering to a subject a liquid volume of blood comprising RBCs that have been obtained by ex-vivo treatment with an activator of the non-canonical Wnt pathway. Transfusing may be performed pursuant to any medically appropriate procedure, such as for the treatment of diseases or disorders associated with blood loss or reduced blood function or with RBC cytoskeleton/membrane disease. Alternatively, transfusing may be performed in anticipation of surgery in order to medical optimize the subject to withstand the rigors associated with surgical stress. Specific methods for administration include those known in the art, such as through use of an intravenous catheter. Specific methods for administration include those known in the art, such as through use of an intravenous catheter. Other contemplated uses, be them therapeutic or non-therapeutic or prophylactic are described hereinbelow.

The administration of the blood cells treated as described herein may be following storage of the blood or immediately following treatment. For example in the latter case, administration of blood may be performed 24 hours, 18 hours, 12 hours, 10 hours, 8 hours, 4 hours, 1 hour, 30 minutes, 15 minutes, 10 minutes, 2 minutes, 1 minute, or less, after incubation of the red blood cells with the activator of the non-canonical Wnt pathway. In some processes, the methods are “point of care,” wherein the methods of the present technology are performed at a location proximate, such as in the same room (for example, bed side) or otherwise immediately adjacent, to the subject to be transfused with the RBCs. Such point-of-care processes may be performed using a system comprising an apparatus adapted to perform two or more sequential steps of a process of the present invention, such as the steps of obtaining blood, adding the activator of the non-canonical Wnt pathway, incubating, washing, and administering. In some embodiments, such a system is in fluid communication with a device, such as an intravenous catheter, for obtaining blood from a subject or administering blood to a subject. In some embodiments, the RBCs administered are autologous.

A subject in need of a transfusion can have a disorder characterized by reduced tissue oxygenation. Such disorders include those wherein when blood flow is fixed, restricted, reduced, or stopped. Furthermore, blood transfusions can be necessary when blood is lost though injury, surgery or disease. Subjects and disorders that may be treated include: subjects with sepsis or septic shock that are anemic and require a blood transfusion; subjects with Upper Gastrointestinal Bleeding (“UGIB”) that are anemic and require a blood transfusion; subjects subjected to severe trauma that are anemic and require a blood transfusion; subjects that are critically ill (adult and pediatric) in an intensive care unit, who are anemic and require a blood transfusion; subjects that undergo open heart surgery and receive a blood cardioplegia solution to perfuse the heart during hypothermic, ischemic cross-clamp, thus providing better oxygenation of the myocardium during open-heart surgery; subjects suffering a stroke, treating ischemic brain tissue following a stroke, thus increasing the oxygen delivery capacity of the systemic circulation via exchange transfusion or by direct administration to the ischemic area via arterial catheter or by retrograde perfusion via the venous circulation; subjects undergoing obstetrical complications, subjects with bleeding ulcers; subjects with hemolytic anemia; subjects with thrombocytopenia, pneumonia and acute respiratory distress.

A non-therapeutic method is also contemplated herein for increasing the overall performance of a treated subject (healthy). For example, some athletes improve their performance by blood doping: first about 1 litre of their blood is extracted, then the red blood cells are isolated, frozen and stored, to be reinjected shortly before the competition. (Red blood cells can be conserved for 5 weeks at −79° C.). The ex vivo treated blood is contemplated therefore for such non-therapeutic indications. It will be appreciated that such treatments also apply for extreme mountain hiking at high altitudes and as such are contemplated herein.

Composition of the invention (activator of the non-canonical Wnt pathway, or RBC obtainable by the treatment with such an activator) may be administered for the treatment of RBC cytoskeleton/membrane diseases. The normal (non-pathological) erythrocyte membrane skeleton is organized as a hexagonal lattice that underlies the cell membrane. Each side of the hexagon consists of a long, flexible spectrin tetramer, constructed by two a- and b-spectrin heterodimers connected head-to-head, with their tails ending at junctional complexes.

The junctional complex of the cytoskeleton is composed of a short actin filament (F-actin protofilament) and various actin-binding proteins, including adducin and tropomodulin. Adducin and tropomodulin function as actin-capping proteins and facilitate the interaction between spectrin and F-actin. This complex providing deformability on one hand and stability on the other which is so crucial for movement in the capillary network. Defects and deficiencies in components of this meshwork lead to fragile membranes and are associated with various hemolytic anemias (Fowler V M. Curr Top Membr. 2013), also included herein under the term “RBC cytoskeleton/membrane disease”.

According to a specific embodiment, the RBC cytoskeleton/membrane disease is selected from the group consisting of spherocytosis, elliptocytosis, pyropoikilocytosis and stomatocytosis.

Compositions (RBCs having been treated according to the present teachings and/or activator of the non-canonical Wnt pathway)for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the composition may be formulated in aqueous solutions, preferably in physiologically compatible buffers such as Hank's solution, Ringer's solution, or physiological salt buffer.

The activator of the non-canonical Wnt pathway or compositions comprising same described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, and may contain formulatory agents such as suspending, stabilizing and/or dispersing agents.

Alternatively, the activator of the non-canonical Wnt pathway or composition comprising same may be in dry particulate form for constitution with a suitable vehicle, e.g., sterile, pyrogen-free water based solution, before use.

Compositions suitable for use in context of some embodiments of the invention include compositions wherein the activator of the non-canonical Wnt pathway or RBCs treated according to the present teachings are contained in an amount effective to achieve the intended purpose. More specifically, a therapeutically effective amount means an amount of active ingredients (e.g. the activator of the non-canonical Wnt pathway or RBCs treated according to the present teachings) effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation or composition used in the methods of the invention, the therapeutically effective amount or dose can be estimated initially from in vitro assays. Toxicity and therapeutic efficacy of the activator of the non-canonical Wnt pathway and compositions comprising the same described herein can be determined by standard laboratory procedures in vitro or experimental animals. The data obtained from these in vitro and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

Activator of the non-canonical Wnt pathway or compositions comprising the same of some embodiments of the invention may, if desired, be presented in a pack or dispenser device, such as an FDA approved kit, which may contain one or more unit dosage forms containing the activator of the non-canonical Wnt pathway or compositions comprising the same. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of therapeutic compositions, which notice is reflective of approval by the agency of the form of the compositions or human or veterinary administration. Such notice, for example, may be of labeling approved by the U.S. Food and Drug Administration for therapeutic compositions or of an approved product insert. Compositions comprising a preparation of the invention formulated in a compatible carrier may also be prepared, placed in an appropriate container, and labeled for treatment of an indicated condition, as is further detailed above. The pack or kit may also comprise additional agents useful in influencing RBC survival and treating conditions as described herein.

Some additional agents suitable for use with the activator of the non-canonical Wnt pathway of the invention, and/or compositions comprising the same, and methods for its use, include, but are not limited to anti-coagulants such as heparin, LMWheparin, plasminogen activator, streptokinase and urokinase, antibiotics, and anti-platelet drugs such as dipyridamole.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, N.Y. (1988); Watson et al., “Recombinant DNA”, Scientific American Books, N.Y.; Birren et al. (eds) “Genome Analysis: A Laboratory Manual Series”, Vols. 1-4, Cold Spring Harbor Laboratory Press, N.Y. (1998); methodologies as set forth in U.S. Pat. Nos. 4,666,828; 4,683,202; 4,801,531; 5,192,659 and U.S. Pat. No. 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., N.Y. (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and U.S. Pat. No. 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Materials and Methods

Reagents: Ca2_-Mg2_—free Dulbecco phosphate buffer saline (PBS) (Sigma D8537). Dulbecco's modified Eagle's medium (DMEM) (GIBCO) supplemented with 10% fetal calf serum (FCS) (Sigma-Aldrich) and 100 U/ml penicillin/streptomycin (1%) (Biological Industries, Kibbutz Beit Haemek, Israel). All blood samples were collected in Vacutainer tubes containing K-EDTA as anticoagulant (BD Vacutainer® Blood Collection Tubes BD Biosciences). Red blood lysis buffer containing 8.3 g ammonium chloride (NH4Cl), 1.0 g potassium bicarbonate (KHCO3), 1.8 ml of 5% EDTA or 37 mg EDTA, filter sterilize through 0.2 um filter and complete to final volume of 1000 ml with dH2O to pH of 7.4. supplemented with protease inhibitors cocktail (Sigma-Aldrich). Poly-lysines (sigma P4832). Antybodies: Monoclonal mouse anti—Actin (MP 69100), rabbit anti alpha Adducin (Abcam 51130), mouse anti Phospho Adducin (ser 927) (cell signaling 05587), rabbit anti phospho Adducin (THR 445) (Santa Cruz 16738), mouse anti-β-catenin (BD Transduction Laboratories 610154), rabbit anti-Dvl2 (Santa Cruz 13974), mouse anti-Dvl1 (Santa Cruz 8025), rabbit anti-Wnt3a (Cell Signaling C64F2), rabbit anti-Wnt5a (Abcam 72583), rabbit anti-Fz (Santa Cruz 9169), rabbit anti-Fz1 (Santa Cruz 130758), rabbit anti-hemoglobin (Santa Cruz 21005), rabbit anti Lamin B1 (abcam 16048), rabbit anti PKC (Santa Cruz 216), mouse anti spectrin (sigma 3396), Anti-rat horseradish peroxidase-conjugated secondary antibodies were obtained from Santa Cruz Biotechnology, Anti-mouse and anti-rabbit secondary antibodies were obtained from Jackson Immuno Research. CytoPainter Phalloidin-iFluor 488 Reagent (abcam 176753), secondary antibodies for IF Alexa red and green (Molecular Probes, Grand Island, N.Y., USA). Kits: RAC1 activation assaybiochem kit (Cytoskeleton BK 035), RhoA activation assay biochem kit (cytoskeleton BK 124), RT² Profiler™ PCR Array Human WNT Signaling Pathway (Qiagen 043Z).

Blood Preparation

All blood samples were collected in Vacutainer tubes containing K-EDTA as anticoagulant, kept at 4° C. and used within 24-48 h. Peripheral blood was centrifuged at 1000×g for 15 min. Supernatant and top layer of the pellet were discarded in order to remove contaminating leucocytes from RBC pellet. Purified RBCs were washed 3 times with PBS and lysed in a lysis buffer (800 ml dH2O supplemented with 8.3 g ammonium chloride (NH4Cl) , 1.0 g potassium bicarbonate (KHCO3), 37 mg EDTA, filter sterilized through 0.2 μm filter and completed to a final volume of 1000 ml with dH2O to pH of 7.4) supplemented with protease inhibitors. The aliquots of the haemolysates were stored at −20° C. before further analysis.

RBC Preparation

To maximize the number of identified RBC proteins, membrane associated proteins, as well as cytoplasmic proteins, were separated. The haemolysates obtained after sedimentation were centrifuged at 45,000×RPM for 45 min at 4° C. to separate the lysed cells and the soluble cytoplasmic proteins. The soluble cytoplasmic proteins were collected and cleared by recentrifugation. Lysed RBCs were further washed with 10 volume of ice-cold lysis buffer to prepare cell membranes. The membrane pellet was directly solubilized in SDS sample buffer.

*The Haemolysates were enriched with over 95% hemoglobin. For hemoglobin depletion the hemoglobin depletion kit of HemoVoid, Biotech support group was used.

Conditioned Media Preparation
The control and the Wnt conditioned media were obtained by using the following cells line:
Control medium obtained from L cells—Mouse fibroblast cells from subcutaneous connective tissue (ATCC No. CRL-2648).
Wnt3a condition medium obtained from L-Wnt3a cells—(L cells stable expressing Wnt3a). The cells secrete biologically active Wnt3a protein (ATCC No. CRL-2647).
Wnt5a condition medium obtained from L-Wnt5a cells—(L cells stable expressing Wnt5a). The cells secrete biologically active Wnt5a protein (ATCC No. CRL-2814).

In some cases, recombinant human Wnt-3a protein (R&D System 5036-WN), and recombinant human Wnt-5a protein (R&D System 645-WN) were used at a concentration of 0.2 ug/mL in PBS.

In each of the following methods RBCs were incubated in conditioned media for 24-48 hours in 37° C. and then collected and analyzed by CBC (count blood cells), western blot or immunofluorescence analysis.

CBC (Complete Blood Count)

In collaboration with the hematology department at Ichilov hospital, the Complete blood count analyzer was used to get number of cells (N) and other essential characteristics of the red blood cell: hemoglobin (HGB) levels, hematocrit (HCT) levels and mean cellular volume (MCV).

Western Blot Analysis

The haemolysates was separated on 7.5% or 10% SDS-polyacrylamide gel electrophoresis (SDS-PAGE), and proteins were transferred to nitrocellulose membranes. After blocking with 5% low fat milk, membranes were incubated with primary antibodies, washed three times with 0.001% Tween-20 in PBS, incubated for 60 minutes with secondary antibodies, washed again three times and exposed to enhanced chemiluminescence (ECL) detection analysis using horseradish peroxidase-conjugated secondary antibodies.

Immunofluorescence

Glass coverslips were coated with 100 μL of 0.1 mg/mL poly-L-lysine (molecular weight, 70-150 kDa) in PBS for 1 hour. Next, 160×10̂6 RBC was applied on the slides and allowed to adhere for 40 minutes at room temperature. Cells were washed with PBS and fixed in PBS containing 4% paraformaldehyde (PFA) for 20 minutes. Fixed cells were washed twice with PBS, permeabilized with PBS containing 0.1% Triton (PBT) for 10 minutes and blocked in PBS containing 1% BSA and 0.1% Triton (BBT) for one hour. Afterwards, cells were incubated at room temperature with primary antibodies for 60 minutes, washed three times with PBT, incubated with secondary antibodies for 30 minutes, and washed again three times. Slides were visualized by confocal microscopy or by phase contrast microscopy (Leica SPS, Leica Microsystems, Bannockburn, Ill.).

RT2 Profiler PCR Wnt Array

The reverse transcription (RT2) Profiler polymerase chain reaction (PCR) Array Human Wnt Signaling Pathway array (Qiagen) was used. Total RNA from fresh RBCs was isolated using the TRIZOL Reagent (Invitrogen) and further purified using miRNeasy Mini Kit (Qiagen). RNA was quantified by Nanodrop ND-1000. 0.5 μg total RNA was reverse transcribed according to the manufacturer's protocol and then real-time PCR was done by adding cDNA directly to PCR Master Mix containing SYBR Green and References Dyes (SuperArray). The mixtures were then aliquoted into 96-well PCR array plates, which profile the expression of 84 Wnt signaling-specific genes plus controls for both human Wnt signaling pathways. Thermal cycling condition was used: 95° C. for 10 minutes, followed by 40 cycles of 95° C. for 15 seconds and 60° C. for 60 seconds. Data analysis and the cycle threshold (CT) values, which were defined as the fractional cycle number at which the fluorescence passes an arbitrarily set threshold.

Statistical Analysis

Results are presented as mean ±SEM. Statistical analysis among groups was performed using Student's t-test. p<0.05 was regarded as statistically significant.

### Example 1

### Effect of Wnt3a and Wnt5a on RBC Survival and Quality

Despite the fact that Wnt-3a and. Wnt5a are not expressed in RBCs, the present inventors examined their effect on RBCs. RBCs (500×10⁶) were incubated with Wnt-3a CM and wnt5a CM at 37° C. for 24 and 48 hours and several parameters indicating health and survival were measured. Surprisingly, treatment of the RBCs in the enriched Wnt-3a or wnt5a CM resulted in increased cell survival (238×10⁶ and 221×10⁶ cells/dish respectively) after 24 hours incubation as compared to control media (100×10⁶ cells/dish). After 48 hours RBCs survived only in the Wnt-3a media (173×10⁶ cells/dish) and Wnt-5a media (180×10⁶ cells/dish). Increased RBC survival in Wnt-3a or wnt5a media was followed by an increase in hemoglobin (HGB) levels, hematocrit (HCT) levels and mean cellular volume (MCV). (FIGS. 1A-C).

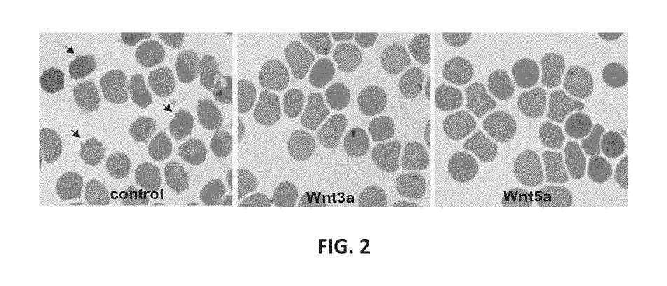

RBC morphology was measured after 24 hours by cytospin, fixation of 13 minute with ethanol and staining with maygreenwald for 13 minute. RBC in control media showed shrunken and fragmented cells to a spheroid-shaped (spheroechinocyte) in addition to various membrane protrusions or spicula (echinocyte). However in RBCs exposed to the Wnt-3a or Wnt-5a CM, these changes were minor and maintained their morphology (FIG. 2).

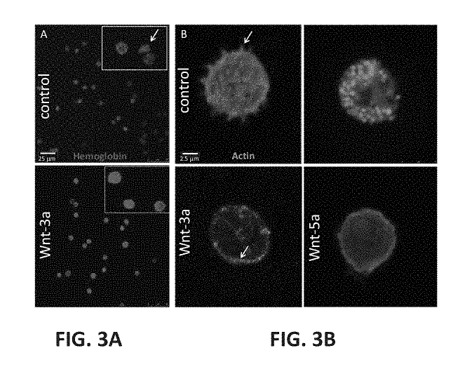

FIGS. 3A-B show that hemoglobin levels and RBCs morphology are affected by Wnt ligands. RBCs treated with Wnt-3a or Wnt-5a CM at 37° C. for 24 h were stained for hemoglobin and actin. RBCs in control media were shrunken, lost their normal shape, characterized by “spiky” in their membrane and lower hemoglobin levels in their cytoplasm in addition to aggregate of actin in cytoplasm. All these effects were moderate in the RBC treated with wnt3a CM in addition to actin localization in the membrane.

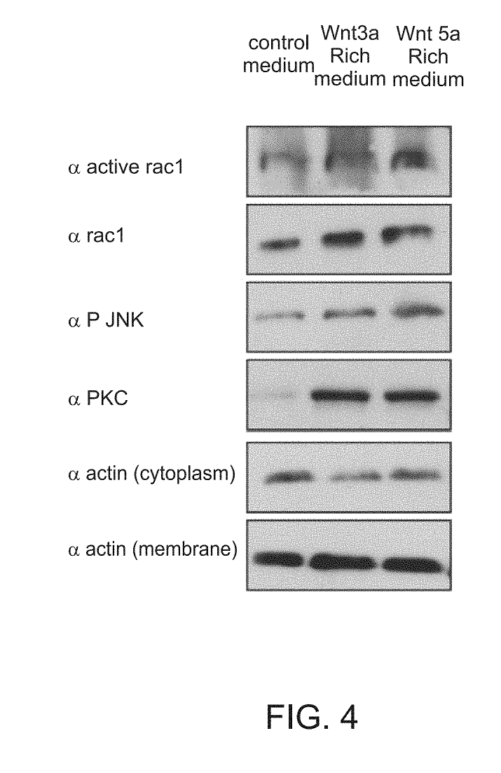

RBC protein expression levels were evaluated after 24 hours of treatment with Wnt CM by western blot assay. The lysate was separated into two fractions: membranal and cytoplasmic. In the membrane fraction active RAC1, phospho-JNK and actin expression levels are higher in RBCs incubated with the Wnt-3a and wnt5a enriched media. In the cytoplasmic fraction, these proteins cannot be found except for actin which is decreased in Wnt-3a and wnt5a media (FIG. 4). The increase in membrane actin and the decrease in cytoplasmic actin after treatment with Wnt CM can support last results which showing that free actin in the cytoplasm shift to the membrane to allow actin polymerization.

Conclusions

The present inventors have found that treating RBCs with Wnt-3a and Wnt-5a increase the survival and efficacy of RBC and alters their protein expression pattern.

The changes include increase in Rac1 and phospho JNK protein levels and a reduction in cytoplasmic actin levels. It is suggested that Wnt-3a and Wnt-5a initiate the non-canonical pathway in RBC leading to the activation of Racl. This activation can affect cells cytoskeleton in two ways: the first by actin polymerization and the second by inhibiting the breaking down of the cytoskeleton complex (actin-spectrin-adducin).

### Example 2

### The Expression Levels of Wnt Signaling Components in RBCs

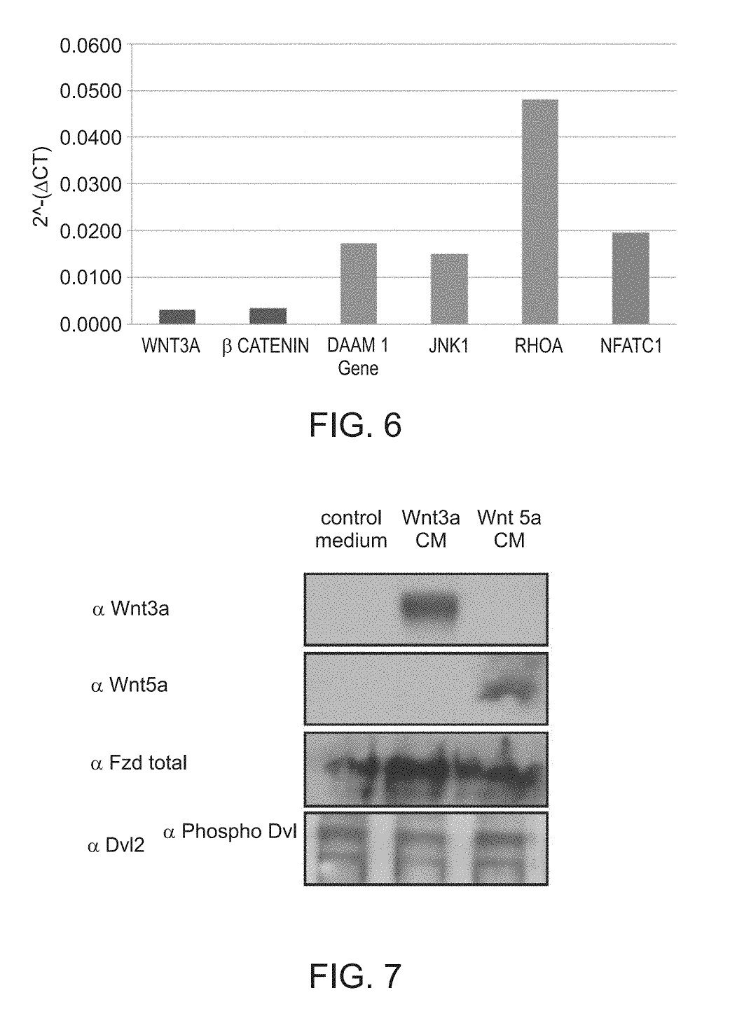

The expression of canonical components and non-canonical components was tested in RBCs by mRNA expression. mRNA expression in RBCs of the canonical pathway components was not detected (β-catenin, Wnt-3a) while the mRNA of the non-canonical components, both PCP (DAAM1, JNK1, RHOA) and calcium pathways (NFATC1), was detected (FIG. 6).

### Example 3

### Upstream Activation of Wnt Signaling in RBC

The upstream activation of the Wnt signaling includes some proteins but mainly depends on Wnt-Fzd binding. RBCs which were treated with Wnt-3a/5a conditioned media demonstrated clear expression of Wnt-3a and Wnt-5a in the membrane fraction at the protein level. Also Fzd receptors were found in these cells. Additionally, DVL2 showed to be expressed in two forms (phosphorylated and unphosphorylated) (FIG. 7).

Clear expression of the receptors and co receptors of the Wnt ligand in reticulocytes was found except for the ligand itself. Eight out of the nine Fzd receptors were identified in the reticulocyte. Fzd 1,3,4 expressed in high levels as compared to the others. LRP6 and Dvl2 were also detected at high levels (FIG. 8).

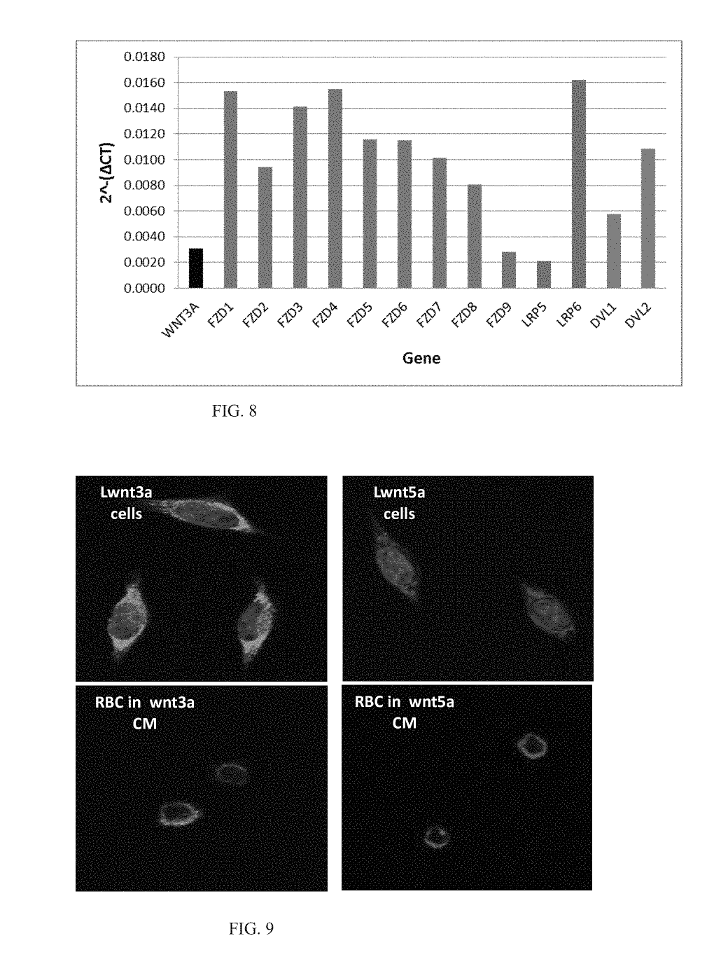

The ability of Wnt ligand to bind the membrane of the RBCs was tested. RBCs were treated with Wnt-3a and Wnt-5a CM. 16 h later the localization of Wnt-3a and Wnt-5a in the erythrocyte membrane was analyzed by using an immunofluorescence assay. Interestingly. anti Wnt-3a strongly stained the plasma membranes of both treated RBC with Wnt-3a CM and the control (Lwnt-3a cells) (FIG. 9 left panel). Same results were obtained for with anti Wnt-5a staining in treated RBC with Wnt-5a CM and the control (Lwnt-5a cells) (FIG. 9 right panel).

### Example 4

### Downstream Activation of Wnt Signaling in RBC

The Downstream activation of the Wnt signaling includes several proteins which their expression in RBC was already showed here (FIGS. 7-9).

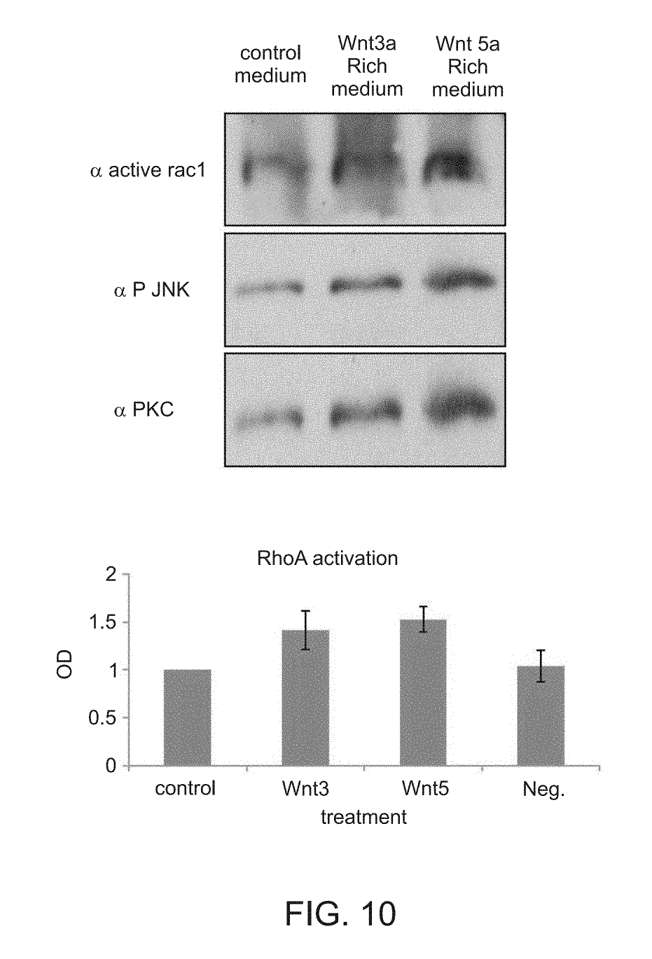

It is suggested that upstream activation through Wnt-Fzd binding in RBC can lead to different expression and phosphorylation changes of these proteins. Treatment with Wnt ligands (both Wnt-3a and Wnt-5a) leads to significant increase in membrane levels of PKC, phospho-JNK, active RAC1-GTP (as detected by using a specific Active Rac1 Detection Kit) and active RhoA (as detected by using a RhoA G-LISA Activation Assay Kit). (FIG. 10).

### Example 5

### Distribution of Actin During Activation of Wnt Signaling in RBC

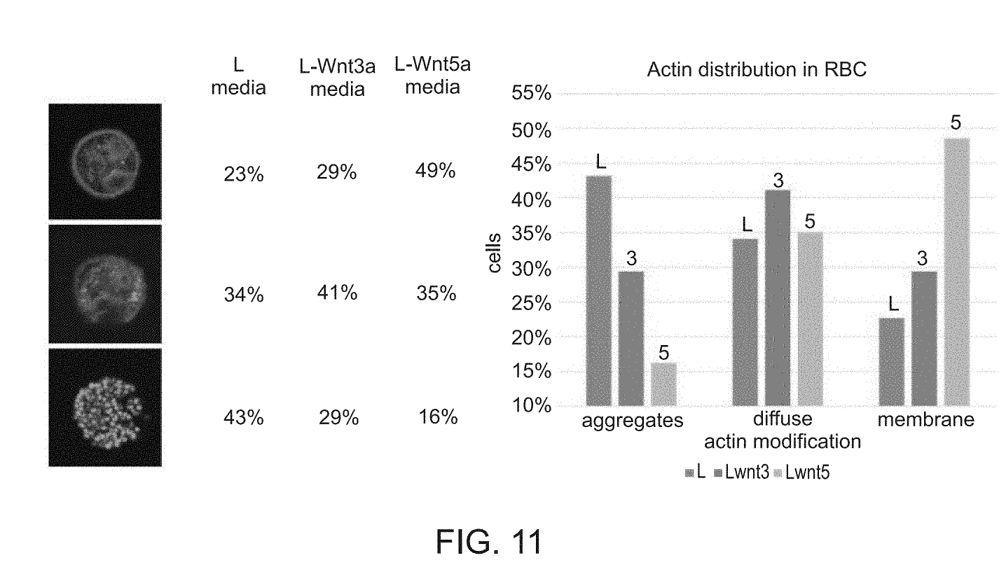

The important molecular features that establish the stability or lability of RBC cytoskeleton is the actin-spectrin-adducin complex, which partly depends on the actin polymerization state. Rhodamine-phalloidin staining of fixed erythrocytes was used in order to test subcellular distribution of actin, shifting of actin from the cytoplasm to membrane or vise versa and the morphology of the cells. Generally, actin appears in the cells in one of these three forms: (A) membrane actin as demonstrate in the cytoskeleton complex (B) cytoplasm actin as demonstrated by diffused actin in the cytoplasm (C) aggregate actin as demonstrated by breakdown of the cytoskeleton. Most of the wnt5a treated cells showed actin localization in the membrane while aggregates of actin mainly appear in control (FIG. 11).

### Example 6

### Actin Polymerization in RBC

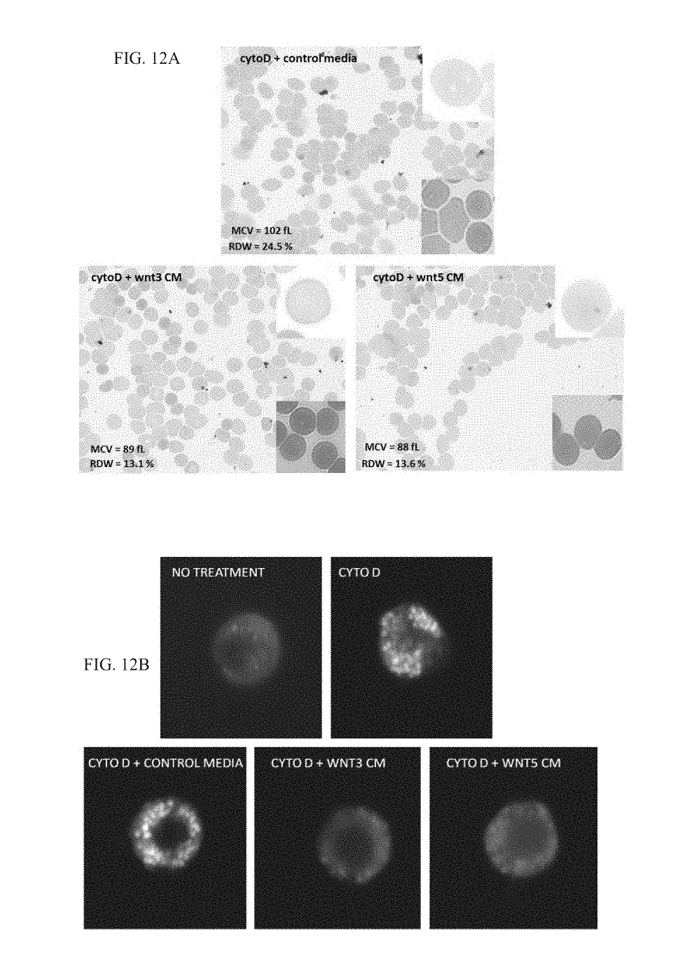

Cytochalasin D (CytoD) is known to inhibit actin polymerization or depolymerization at barbed ends. The recovery effects of Wnts CM in RBCs was tested after incubation with CytoD in order to evaluate the potential effect of Wnt3/5 on polymerized actin in RBCs (FIGS. 12A-B). As show in FIGS. 12A-B, RBCs incubated with a medium containing Cyto D for 3 h, and then with Wnt 3/5 CM (in the absence of CytoD) for 6 h, were stained using two different methods. Both methods showed significant effects of Wnt3a and Wnt5a compared to control media; Wright gimesa stain demonstrates the effect of CytoD—cells varied in size including some fragmented cells and many cells lost their membrane and cytoplasm shape characterized by slight white holes. This effect was dramatically attenuated in cells treated with Wnt-3a and Wnt-5a CM (FIG. 12A). Immunofluorescence analysis (FIG. 12B) demonstrates aggregates of cytoplasm actin following treatment with CytoD. Recovering the cells with Wnt-3a and Wnt-5a caused rapid remodeling of the actin cytoskeleton. Treatment with Wnt3a/5a also abolished almost completely the effect of cytoD on aggregation of actin and cytoplasm conformation. (FIG. 12B).

### Example 7

### Regulation of the RBC Cytoskeleton Membrane During Activation of the wnt Signaling in RBC

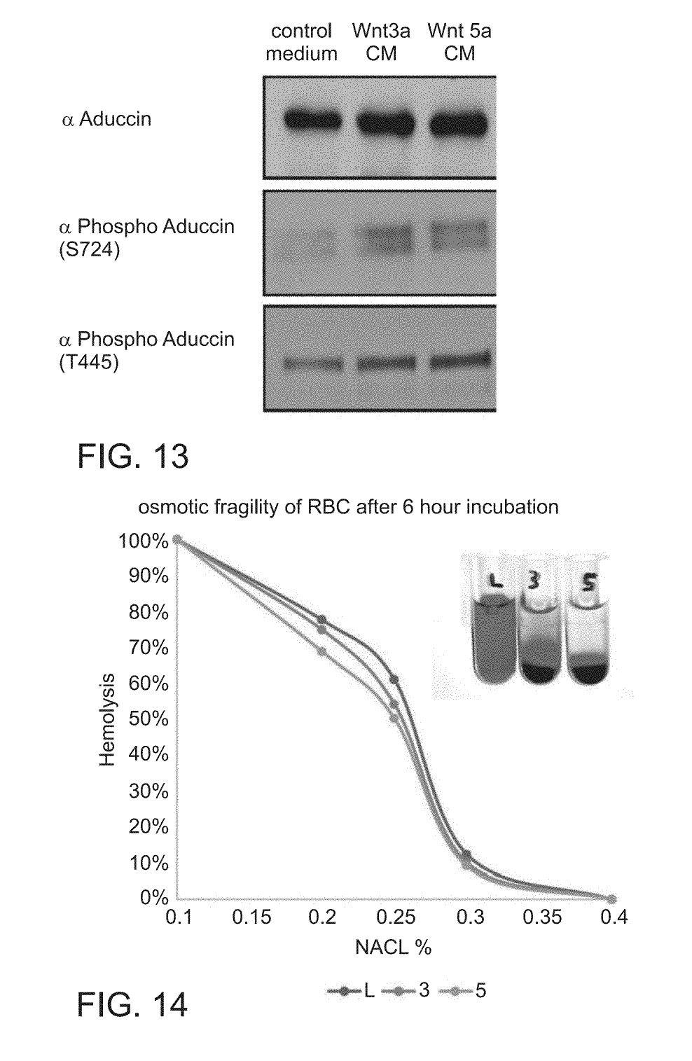

Previous studies reveal the structure and dynamics of actin filament capping are mostly based on the capping protein adducin, which can be phosphorylated on serine-724 (Ser724) by PKC or on Thr 445 by RhoA. These phosphorylation events control the erythrocyte cytoskeleton formation. Treating RBCs with Wnts CMs leads to activation of PKC and RhoA resulting in phosphorylation of adducin which in turn can increase the cells membrane flexibility and improve the vitality of the cell (FIG. 13).

### Example 8

### Red Blood Cells Functions

Osmotic fragility is a blood test that detects whether red blood cells are more likely to break down. Such a test can also indicate on RBC vitality. It was found that Wnt5a increases RBC stability and decrease their fragility in a hypotonic solution (FIG. 14).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An ex-vivo method of increasing survival of red blood cells (RBCs), the method comprising contacting the RBCs with an activator of the non-canonical Wnt pathway, which results in actin polymerization, thereby increasing survival of red blood cells (RBCs).

2. A population of cells obtainable according to the method of claim 1.

3. A method of storing RBCs, the method comprising contacting the RBCs with an activator of the non-canonical Wnt pathway, which results in actin polymerization, thereby storing red blood cells (RBCs).

4. (canceled)

5. An article of manufacture comprising a blood container comprising an activator of the non-canonical Wnt pathway, which results in actin polymerization.

6. (canceled)

7. The population of cells of claim 2, further comprising a preservative and/or an anticoagulant.

8. The method of claim 1, wherein said RBCs are comprised in whole blood.

9. The method of claim 1, wherein said RBCs are comprised in a population of cells comprising platelets.

10. The method of claim 1, wherein said RBCs are comprised in a population of cells devoid of platelets.

11. The method of claim 1, wherein said RBCs are comprised in a population of cells devoid of nucleated blood cells.

12. The method of claim 1, wherein said RBCs are comprised in a population of cells devoid of bone marrow cells.

13. The method of claim 12, wherein said bone marrow cells comprise mesenchymal stem cells.

14. The method of claim 1, wherein said RBCs are a pure population of cells (i.e., substantially_100% of the contacted cells are RBCs).

15. The method of claim 1, wherein said RBCs comprise irradiated RBCs.

16. (canceled)

17. The method of claim 1, wherein said component of the non-canonical Wnt pathway comprises Wnt.

18. The method of claim 17, wherein said Wnt is selected from the group consisting of Wnt1, Wnt2, Wnt2B, Wnt4, Wnt5A, Wnt5B, Wnt6, Wnt7A, Wnt7B, Wnt8A, Wnt8B, Wnt9A, Wnt9B, Wnt10A, Wnt10B, Wnt11 and Wnt16.

19. The method of claim 17, wherein said Wnt is Wnt5A.

20. The article of manufacture of claim 5, wherein said blood container is a blood bag.
